(19) United States
(12) Patent Application Publication
(10) Pub. No.: US 2011/0287274 A1
(43) Pub. Date: Nov. 24, 2011

(54) RADIATION CURABLE COATING COMPOSITIONS COMPRISING DIACRYLATES

(75) Inventors: Darijo Mijolovic, Mannheim (DE); Sebastien Garnier, Berlin (DE); Reinhold Schwalm, Wachenheim (DE)

(73) Assignee: BASF SE, Ludwigshafen (DE)

(21) Appl. No.: 13/147,039

(22) PCT Filed: Feb. 23, 2010
(86) PCT No.: PCT/EP10/52227

§ 371 Date: Jul. 29, 2011

(30) Foreign Application Priority Data
Mar. 4, 2009 (EP) 09154282.9

(51) Int. Cl. C09D 135/02; B32B 27/36; C08F 2/46; C08F 2/50; C09D 133/14; C07C 69/75

(52) U.S. Cl. 428/521; 524/854; 524/853; 560/127; 427/487; 427/517

(57) ABSTRACT

Process for producing protective or surface coatings by coating of substrates with a radiation curable coating composition and subsequent radiation curing, wherein the radiation curable coating composition comprises a compound of the formula I

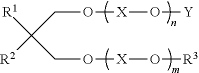

in which
R¹ and R² independently of one another are an organic radical having in each case at least one C atom, but where the sum of the C atoms of R¹ and R² is at least 4, or R¹ and R² together form a ring system comprising at least 4 C atoms,
R³ is a hydrogen atom or Y,
X is a C₁ to C₁₀ alkylene group,
n and m independently of one another are 0 or an integer from 1 to 50,
Y is a group selected from
—CH═CH₂; —CH₂—CH═CH₂;
or

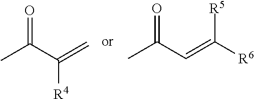

and
R⁴ can be an H atom, a methyl group or —CH₂—C(═O)—OH, and
R5 is an H atom and R6 is a —C(═O)—OH group or, alternatively,
R6 is an H atom and R5 is a —C(═O)—OH group.

The invention relates to a process for producing protective or surface coatings by coating of substrates with a radiation curable coating composition and subsequent radiation curing, wherein the radiation curable coating composition comprises a compound of the formula I

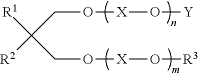

in which
R¹ and R² independently of one another are an organic radical having in each case at least one C atom, but where the sum of the C atoms of R¹ and R² is at least 4, or R¹ and R² together form a ring system comprising at least 4 C atoms,
R³ is a hydrogen atom or Y,
X is a C₁ to C₁₀ alkylene group,
n and m independently of one another are 0 or an integer from 1 to 50,
Y is a group selected from

### —CH═CH₂; —CH₂—CH═CH₂;

or

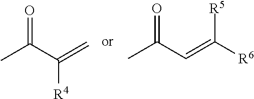

and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

Radiation curable coating compositions possess a range of performance advantages. The constitution of such coating compositions can be chosen so that even without solvent they are liquid at room temperature. In that case, removal of solvent after radiation curing has taken place is no longer necessary.

The viscosity of the coating composition can be adjusted through the nature and amount of radiation curable compounds of low molecular mass, also referred to as reactive diluents, and exemplified by monoacrylates or diacrylates.

A known constituent of radiation curable coating compositions is neopentylglycol diacrylate, a diacrylate of formula I above, but where R¹ and R² are each a methyl group. DE-A 2003 132 discloses coating compositions which comprise neopentylglycol diacrylate or else, for example, 2-ethyl-2-methyl-1,3-propanediol diacrylate (corresponding to formula I but with R¹=ethyl and R²=methyl). Subject matter of US 2008/0038570 are radiation curable printing inks. The printing inks comprise di(meth)acrylates. One di(meth)acrylate referred to is 2-butyl-2-ethyl-1,3-propanediol diacrylate (corresponding to formula I but with R¹=butyl and R²=ethyl). Protective coatings, surface coatings or other use of radiation curable coating compositions is not specified.

Radiation curable coating compositions which are used for protective or surface coatings are required to fulfill a wide variety of performance properties.

The coatings obtained are required in particular to have high hardness and hence to be resistant to mechanical influences. The coatings are also required to be highly resistant to chemicals, solvents or greases, so that the visual appearance is not impaired. Also required is good elasticity or flexibility, so that mechanical loads, such as stress states, do not result in cracks in the coating.

The desire is therefore for coating materials which fulfill the above performance properties as well as possible; in this context it is often problematic to find coating compositions which combine high hardness with the elasticity needed for the particular application. Furthermore, the coating compositions are required to have good processing and coating properties; a prerequisite for such properties, in particular, is a low viscosity.

It was an object of the present invention to provide such coating compositions.

Found accordingly has been the above-defined process for producing protective and surface coatings. Also found have been coating compositions which are particularly suitable for such processes. Furthermore, suitable radiation curable compounds have been found which are used advantageously in the coating compositions.

### The Compounds of Formula I

The process of the invention uses a radiation curable coating composition which comprises a compound of the formula I

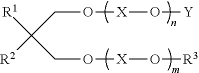

in which
R¹ and R² independently of one another are an organic radical having in each case at least one C atom, but where the sum of the C atoms of R¹ and R² is at least 4, or R¹ and R² together form a ring system comprising at least 4 C atoms,
R³ is a hydrogen atom or Y,
X is a C₁ to C₁₀ alkylene group,
n and m independently of one another are 0 or an integer from 1 to 50,
Y is a group selected from

### —CH═CH₂; —CH₂—CH═CH₂;

or

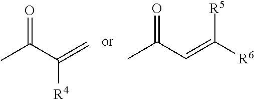

and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

X is preferably a C₁ to C₁₀ alkylene group, with X—O being an alkylene oxide radical of the following alkylene oxides: ethylene oxide, propylene oxide, 1,2-butylene oxide, 2,3-butylene oxide, isobutylene oxide, 1,2-pentane oxide, cyclohexane oxide or styrene oxide. More preferably X—O is the alkylene oxide radical of ethylene oxide or propylene oxide, and preferably, therefore, X is an ethylene or propylene group.

n and m are preferably O or a value from 1 to 20.

In one particularly preferred embodiment n and m are 0, i.e., there are no alkylene oxide groups (X—O—) present.

R³ is a hydrogen atom or one of the above groups; more particularly R³ is a hydrogen atom or group of the formula

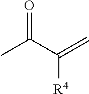

(also represented in an alternative mode as C(═O)—C(R⁴)═CH₂),
where R⁴ is preferably an H atom or a methyl group.

If R⁴ is an H atom, the group is an acryloyl group; if R⁴ is a methyl group, the group is a methacryloyl group.

In accordance with the invention R¹ and R² independently of one another are an organic radical having in each case at least one C atom, but where the sum of the C atoms of R¹ and R² is at least 4, or R¹ and R² together form a ring system comprising at least 4 C atoms.

The sum of the C atoms of R¹ and R² is preferably 4 to 30, more particularly 4 to 25 or 4 to 20, and very preferably 4 to 10.

R¹ and R² may also comprise heteroatoms such as oxygen, nitrogen, sulfur or halogens; in one preferred embodiment R¹ and R² are hydrocarbon groups which comprise no heteroatoms.

In one embodiment, which, like the other embodiments set out below, is preferred, R¹ and R² are alkyl groups, and may be linear or branched.

A compound of the formula I with R¹=butyl and R²=ethyl is already known from the prior art. A radiation curable coating composition including such a compound is unnecessary in the context of this invention and can therefore, preferably, be excluded.

Examples of compounds having linear alkyl groups as radicals R¹ and R² include the following:
2-pentyl-2-propyl-1,3-propanediol diacrylate
   (PPPD-DA with R¹=pentyl, R²=propyl, n,m=0, R³═Y, Y=acryloyl)
2-butyl-2-ethyl-1,3-propanediol diacrylate
   (for short BEPD-DA with R¹=butyl, R²=ethyl, n,m=0, R³═Y, Y=acryloyl)

Y is preferably a group of the formula C(═O)—C(R⁴)═CH₂; if R⁴ is H, the group is an acryloyl group; if R⁴ is methyl, the group is a methacryloyl group.

More preferably R³ is an acryloyl group or a methacryloyl group.

The compound of the formula I is therefore more particularly a diacrylate or dimethacrylate of the following formula II,

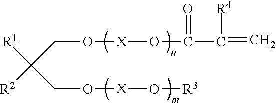

in which X, R¹, R², R⁴, and n and m have the above definitions and preferred definitions.

Particularly preferred radiation curable coating compositions accordingly comprise a compound of the formula II.

Described below are further particular embodiments which are preferred in the context of the present patent specification. Where applicable, the above remarks concerning preferred definitions of the variables also apply to these embodiments below.

### PARTICULAR EMBODIMENTS

In one particular embodiment the compound of the formula I is the compound

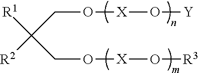

in which
R¹ and R² independently of one another are an organic radical having in each case at least one C atom, but where the sum of the C atoms of R¹ and R² is at least 4, and R¹ or R², or R¹ and R², comprises or comprise at least one tertiary or quaternary carbon atom,
R³ is a hydrogen atom or Y,
X is a C₁ to C₁₀ alkylene group,
n and m independently of one another are 0 or an integer from 1 to 50,
Y is a group selected from

### —CH═CH₂; —CH₂—CH═CH₂;

or

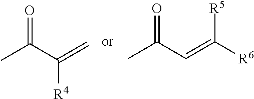

and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

At least one of the radicals R¹ and R² necessarily comprises a tertiary or quaternary C atom. It may be one of the groups R¹ and R², or else both groups R¹ and R², that comprise at least one tertiary or quaternary carbon atom. A tertiary carbon atom is defined as a carbon atom with only one hydrogen atom and 3 bonds to adjacent carbon atoms; a quaternary carbon atom is a carbon atom without a hydrogen atom and with 4 bonds to adjacent carbon atoms. The presence of a tertiary or quaternary carbon atom therefore means that there is a branch in the molecular group. Thus the branched isopropyl group has a tertiary carbon atom, whereas the linear n-propyl group comprises no tertiary or quaternary carbon atom.

In this embodiment as well the compounds are preferably monoacrylates or diacrylates or the corresponding monomethacrylates or dimethacrylates, i.e., compounds of the formula II

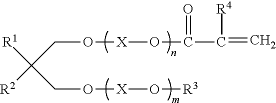

in which
R¹ and R² independently of one another are an organic radical having in each case at least one C atom, but where the sum of the C atoms of R¹ and R² is at least 4 and R¹ or R², or R¹ and R², comprises or comprise at least one tertiary or quaternary carbon atom,
R³ is a hydrogen atom or Y,
X is a C₁ to C₁₀ alkylene group,
n and m independently of one another are 0 or an integer from 1 to 50,
Y is a group —C(═O)—C(R⁴)═CH₂,
and R⁴ can be an H atom or a methyl group.

Compounds contemplated include, for example,
2-isopropyl-2-methyl-1,3-propanediol diacrylate
   (IMPD-DA with R¹=isopropyl, R²=methyl, n,m=0, R³═Y, Y=acryloyl)
2-isopropyl-2-(3-methylbutyl)-1,3-propanediol diacrylate
   (IMBPD-DA with R¹=isopropyl, R²=3-methylbutyl, n,m=0, R³═Y, Y=acryloyl)
2-(2-methylbutyl)-2-propyl-1,3-propanediol diacrylate
   (MBPPD-DA with R¹=2-methylbutyl, R²=propyl, n,m=0, R³═Y, Y=acryloyl).

In another particular embodiment the compound is a compound of the formula I

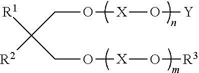

in which
R¹ and R² independently of one another are an organic radical having in each case at least one C atom, where at least one of the radicals, R¹ or R², is or comprises a ring system,
R³ is a hydrogen atom or Y,
X is a C₁ to C₁₀ alkylene group,
n and m independently of one another are 0 or an integer from 1 to 50,
Y is a group selected from

### —CH═CH₂; —CH₂—CH═CH₂;

or

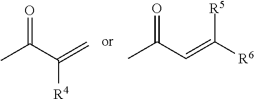

and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

In this embodiment as well the compounds are preferably monoacrylates or diacrylates or the corresponding monomethacrylates or dimethacrylates, i.e., compounds of the formula II

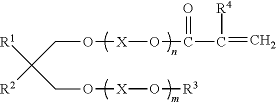

in which
R¹ and R² independently of one another are an organic radical having in each case at least one C atom, where at least one of the radicals, R¹ or R², is or comprises a ring system,
R³ is a hydrogen atom or Y,
X is a C₁ to C₁₀ alkylene group,
n and m independently of one another are 0 or an integer from 1 to 50,
Y is a group —C(═O)—C(R⁴)═CH₂,
and R⁴ can be an H atom or a methyl group.

More particularly the ring system is a phenyl group or cyclohexyl group, and the C atoms of the ring system may carry further substituents, examples being alkyl groups.

Preferably only one of the radicals is a ring system, more particularly a phenyl group or cyclohexyl group, and the other radical is an alkyl group.

Examples include the following:
2-methyl-2-phenyl-1,3-propanediol diacrylate
   (MPPD-DA with R¹=methyl, R²=phenyl, n, m=0).

In another particular embodiment the compound is a compound of the formula I

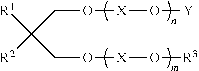

in which
R¹ and R² together form a ring system comprising at least 4 C atoms, preferably comprising 4 to 10 C atoms, more particularly comprising 5 to 8 C atoms, e.g., comprising 5 or 6 or 8 C atoms,
R³ is a hydrogen atom or Y,
X is a C₁ to C₁₀ alkylene group,
n and m independently of one another are 0 or an integer from 1 to 50,
Y is a group selected from

### —CH═CH₂; —CH₂—CH═CH₂;

or

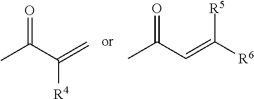

and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

In this embodiment as well the compounds are preferably monoacrylates or diacrylates or the corresponding monomethacrylates or dimethacrylates, i.e., compounds of the formula II

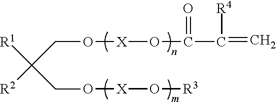

in which
R¹ and R² together form a ring system comprising at least 4 C atoms, preferably comprising 4 to 10 C atoms, more particularly comprising 5 to 8 C atoms, e.g., comprising 5 or 6 or 8 C atoms,
R³ is a hydrogen atom or Y,
X is a C₁ to C₁₀ alkylene group,
n and m independently of one another are 0 or an integer from 1 to 50,
Y is a group —C(═O)—C(R⁴)═CH₂,
and R⁴ may be an H atom or a methyl group.

Examples include the compound having the following formula:

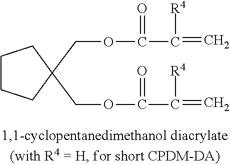

or the compound having the following formula:

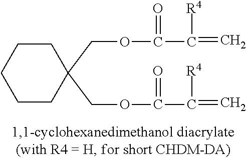

or the compound having the following formula:

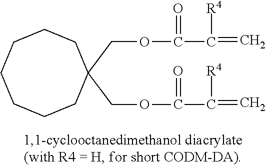

### Preparation of the Compounds of the Formula I

The compounds of the formula I can be prepared in a simple way starting from the known diols.

The presence of alkylene oxide groups (X—O—), where desired, can be brought about in a simple way by alkoxylation of these diols with alkylene oxides, more particularly with ethylene oxide or propylene oxide; n and m in formulae above are then not 0. For the alkoxylation, the diol may be reacted in the presence of suitable catalysts with the desired amount of alkylene oxide, more particularly ethylene oxide or propylene oxide or a mixture of the two. Catalysts used may be alkali metal or alkaline earth metal hydroxides, Lewis-acidic catalysts, or what are known as double metal cyanide catalysts, as described in DE 102 43 361 A1 and the references cited therein.

Depending on the definition of the variables Y, the compounds of the formula I may be obtained by reacting the diols or alkoxylated diols with a further compound.

The further compound may preferably be

acrylic acid or methacrylic acid, their acid halides or alkyl esters, if Y is the acryloyl or methacryloyl group

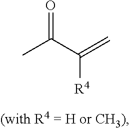

acetylene or another vinylating agent, if Y is the —CH═CH₂ group,
itaconic acid, itaconic acid dialkyl esters, if Y is

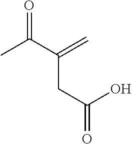

and
maleic acid, maleic anhydride, maleoyl dichloride, maleic acid dialkyl esters or fumaric acid, fumaroyl dichloride, fumaric dialkyl esters, preferably maleic anhydride, if Y is the group

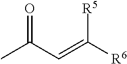

and
R⁵ is an H atom and R⁶ is a C(═O)—OH group or, alternatively,
R⁶ is an H atom and R⁵ is a C(═O)—OH group, and
Allyl chloride or another allylating agent, if Y is the —CH₂—CH═CH₂ group.

The above reactions are esterifications (reaction with acrylic acid, acryloyl halides, acrylic esters, methacrylic acid, methacroyl halides, methacrylic esters, itaconic acid, itaconic acid dialkyl esters, maleic acid, maleic anhydride, maleoyl dichloride, maleic acid dialkyl esters or fumaric acid, fumaroyl dichloride, dialkyl esters of fumaric acid), vinylations or allylations, which can be carried out by the skilled worker simply and in a known way.

Depending on the amounts of the diols and/or alkoxylated diols and of the further compound that are used it is possible for monoacrylates, monomethacrylates, monovinyl compounds, monoallyl compounds, and monoesters of itaconic acid, maleic acid or fumaric acid (mono compounds for short) or diacrylates, dimethacrylates, divinyl compounds, diallyl compounds or diesters of maleic acid or fumaric acid (di compounds for short), or mixtures of the mono and di compounds, of the formula I to be obtained.

In order to obtain exclusively or predominantly di compounds, the other compound used ought to be employed in an amount at least equivalent to the hydroxyl groups of the diol.

In many cases, mixtures of mono and di compounds that are obtained can be used in radiation curable compositions without further working-up or separation of the compounds.

### The Constitution of the Radiation Curable Coating Composition

The radiation curable coating composition may be composed exclusively of one compound of the formula I or of a mixture of such compounds. In particular, in addition to compounds of the formula I, the radiation curable coating composition may comprise other radiation curable compounds or else thermally curable compounds, and also other additives.

The radiation curable coating composition is composed preferably to an extent of 0.1% to 50% by weight, more particularly 0.5% to 40% by weight, of compounds of the formula I. In one especially preferred embodiment the amount of compounds of the formula I in the coating composition is at least 1% by weight and more particularly at least 5% by weight.

The percentages by weight are based on the overall coating composition.

Contemplated more particularly as other radiation curable compounds are other compounds having at least one acryloyl or methacryloyl group ((meth)acryloyl group for short); preferred compounds of this kind are set out below under “Monomers” or “Crosslinkers”. The radiation curable coating composition is composed in one preferred embodiment to an extent of more than 50% by weight, more preferably to an extent of more than 70% by weight, of compounds having at least one (meth)acryloyl group, which may be solely compounds of the formula I or else compounds of the formula I and other compounds having at least one (meth)acryloyl group.

Further constituents contemplated which, after curing has taken place, together form the polymer film are, in addition to the compounds of the formula I, other monomers, crosslinkers or other compounds, such as polymers, for example. The compounds of the formula I and, where used, the further monomers, crosslinkers or other compounds are also referred to collectively as binders.

### Further Monomers

Further monomers (compounds having a copolymerizable, ethylenically unsaturated group) preferably have a molar weight of less than 300, more particularly less than 200, g/mol. They serve in particular as reactive diluents. Possible monomers are selected, for example, from C₁-C₂₀ alkyl (meth)acrylates, vinyl esters of carboxylic acids comprising up to 20 C atoms, vinylaromatics having up to 20 C atoms, ethylenically unsaturated nitriles, and vinyl ethers of alcohols comprising 1 to 10 C atoms.

In particular, mixtures of the (meth)acrylic esters are suitable.

Vinyl esters of carboxylic acids having 1 to 20 C atoms are, for example, vinyl laurate, vinyl stearate, vinyl propionate, vinyl esters of Versatic acid, and vinyl acetate.

Vinylaromatic compounds contemplated include vinyltoluene, α-methylstyrene, 4-n-butylstyrene, 4-tert-butylstyrene, and—preferably—styrene.

Examples of nitriles are acrylonitrile and methacrylonitrile.

Examples of vinyl ethers include vinyl methyl ether, vinyl ethyl ether, n-butyl vinyl ether, isobutyl vinyl ether, tert-butyl vinyl ether, cyclohexyl vinyl ether, 4-hydroxybutyl vinyl ether or C₅-C₂₀ n-alkyl vinyl ethers. Preference is given to vinyl ethers of alcohols comprising 1 to 4 C atoms.

Other vinyl compounds include, for example, N-vinylimidazole, alkyl-substituted N-vinylimidazoles, N-vinylformamide, N-vinyl-2-pyrrolidone, N-vinylcaprolactam, and aminopropyl vinyl ethers.

Preferred monomers are, generally, (meth)acrylate compounds, and more particularly the C₁ to C₂₀ alkyl acrylates and methacrylates, more particularly C₁ to C₈ alkyl acrylates and methacrylates.

Especially preferred are methyl acrylate, ethyl acrylate, n-butyl acrylate, n-hexyl acrylate, octyl acrylate, and 2-ethylhexyl acrylate, and also mixtures of these monomers.

Also contemplated as monomers, furthermore, are polar monomers with isocyanate, amino, amide, epoxy, hydroxyl or acid groups.

Examples include monomers with carboxylic, sulfonic or phosphonic acid groups (e.g., vinylphosphonic acid). Carboxylic acid groups are preferred. Examples include acrylic acid, methacrylic acid, itaconic acid, maleic acid or fumaric acid.

Further monomers are also, for example, monomers comprising hydroxyl groups, more particularly C₁ to C₁₀ hydroxyalkyl (meth)acrylates, (meth)acrylamide, and monomers comprising ureido groups, such as ureido (meth)acrylates.

Further monomers also include mono(meth)acrylates which comprise alkoxyl groups. These monomers are readily obtainable by alkoxylation of a monoalcohol, examples being C₁ to C₁₀ alkanols, with ethylene oxide and/or propylene oxide, and subsequent etherification with (meth)acrylic acid.

Also contemplated are reaction products of (meth)acrylic acid and monoepoxides, e.g., phenyl glycidyl ether or Versatic acid glycidyl ether.

Further monomers additionally include phenyloxyethylglycol mono(meth)acrylate, glycidyl acrylate, glycidyl methacrylate, amino (meth)acrylates such as 2-aminoethyl (meth)acrylate, or N-vinyl-N-methylacetamide.

### Crosslinkers

The term “crosslinker” refers here to compounds having more than one copolymerizable, ethylenically unsaturated group; the crosslinkers are preferably of low molecular mass and more particularly have a molar weight of less than 5000 (Mw in the case of defined individual compounds) or, in the case of mixtures, of a weight-average molar weight of less than 5000.

Compounds contemplated include more particularly compounds having at least two ethylenically unsaturated, free-radically or ionically polymerizable groups (polymerizable group for short). Preference is given to compounds having (in the case of mixtures on average) from 2 to 6, more preferably 2 to 4, polymerizable groups. The above polymerizable group may be, for example, N-vinyl groups, vinyl ether groups or vinyl ester groups, and more particularly are acryloyl or methacryloyl groups ((meth)acryloyl groups for short).

The weight-average molecular weight Mw of the crosslinkers is preferably below 5000, more preferably below 3000, g/mol (determined by gel permeation chromatography with polystyrene as standard and with tetrahydrofuran as eluent).

The crosslinkers are more particularly (meth)acrylic compounds.

They may be, for example, (meth)acrylates, i.e., esters of acrylic acid or methacrylic acids.

(Meth)acrylates include (meth)acrylic esters and more particularly acrylic esters of polyfunctional alcohols, more particularly those which apart from the hydroxyl groups comprise no other functional groups or at most ether groups. Examples of such alcohols are, for example, difunctional alcohols, such as ethylene glycol, propylene glycol, and their counterparts with higher degrees of condensation, such as, for example, diethylene glycol, triethylene glycol, dipropylene glycol, tripropylene glycol, etc., butanediol, pentanediol, hexanediol, neopentylglycol, alkoxylated phenolic compounds, such as ethoxylated and/or propoxylated bisphenols, cyclohexanedimethanol, alcohols with a functionality of three or more, such as glycerol, trimethylolpropane, butanetriol, trimethylolethane, pentaerythritol, ditrimethylolpropane, dipentaerythritol, sorbitol, mannitol, and the corresponding alkoxylated alcohols, more particularly ethoxylated and propoxylated alcohols.

Methacrylate compounds further include polyether (meth)acrylates, which are (meth)acrylic esters of polyetherols. Polyetherols are obtainable in a known way by reaction of polyhydric alcohols, more particularly, for example, the above alcohols, with alkylene oxides, more particularly ethylene oxide or propylene oxide. The degree of alkoxylation per hydroxyl group is preferably 0 to 10, i.e., 1 mol of hydroxyl group may be alkoxylated preferably with up to 10 mol of alkylene oxides.

(Meth)acrylate compounds further include polyester (meth)acrylates, which are the (meth)acrylic esters of polyesterols.

Examples of polyesterols contemplated include those of the kind which may be prepared by esterification of polycarboxylic acids, preferably dicarboxylic acids, with polyols, preferably diols. The starting materials for hydroxyl-containing polyesters of this kind are known to the skilled worker. As dicarboxylic acids it is possible with preference to use succinic acid, glutaric acid, adipic acid, sebacic acid, o-phthalic acid, terephthalic acid, isophthalic acid, their hydrogenation products, and esterified derivatives, such as anhydrides or dialkyl esters of the stated acids. Also contemplated are maleic acid, fumaric acid, tetrahydrophthalic acid or their anhydrides. Polyols contemplated include the abovementioned alcohols, preferably ethylene glycol, 1,2- and 1,3-propylene glycol, 1,4-butanediol, 1,6-hexanediol, neopentylglycol, 2-butyl-2-ethyl-1,3-propanediol, cyclohexanedimethanol, and also polyglycols of the ethylene glycol and propylene glycol types.

Polyester (meth)acrylates can be prepared in two or more stages or else in one stage, as described in EP 279 303, for example, from acrylic acid, polycarboxylic acid, and polyol.

In one specific embodiment of the polyester (meth)acrylates they may be (meth)acrylates of polycaprolactones or polycarbonatediols.

The compounds may further be, for example, epoxide (meth)acrylates or urethane (meth)acrylates.

Epoxide (meth)acrylates are those, for example, of the kind obtainable by reaction of epoxidized olefins or poly- and/or mono- or diglycidyl ethers, such as bisphenol A diglycidyl ether, with (meth)acrylic acid.

The reaction is known to the skilled worker and described for example in R. Holmann, U.V. and E.B. Curing Formulation for Printing Inks and Paints, London 1984.

Urethane (meth)acrylates are more particularly reaction products of hydroxyalkyl (meth)acrylates with poly- and/or diisocyanates (see likewise R. Holmann, U.V. and E.B. Curing Formulation for Printing Inks and Paints, London 1984).

The above (meth)acrylate compounds may each also comprise functional groups, hydroxyl groups for example, which are not esterified with (meth)acrylic acid.

Examples of further crosslinkers include unsaturated polyesters of low molecular mass, which have double bonds, as a result in particular of the presence therein of maleic acid or fumaric acid, and are copolymerizable.

Further crosslinkers are also, for example, vinyl esters of dicarboxylic acids having 1 to 20 C atoms, examples being divinyl succinate, divinyl adipate, divinyl cyclohexyl-1,4-dicarboxylate, and divinyl terephthalate.

Further crosslinkers are also, for example, divinyl ethers such as di-, tri-, or tetraethylene glycol divinyl ether, butanediol divinyl ether or cyclohexanedimethanol divinyl ether.

Preferred crosslinkers are liquid at 20° C. and 1 bar.

In one preferred embodiment the coating composition comprises (meth)acrylic compounds, more particularly (meth)acrylic esters of polyfunctional alcohols, more particularly those which apart from the hydroxyl groups comprise no other functional groups, or at most ether groups, more particularly (meth)acrylic compounds which are liquid at 20° C. and 1 bar and have 2 to 4 (meth)acryloyl groups.

### Other Compounds

Other compounds which may be a constituent of the binder are, for example, polymers. Suitable polymers may possess reactive groups, examples being polymerizable groups or functional groups, so that, on curing, there is attachment to the compounds of the formula I, the above other monomers or crosslinkers. Also contemplated, however, are polymers without such groups, which in the resultant coating then form an independent continuous phase or an interpenetrating network.

Suitable polymers are, for example, polyesters, polyadducts, more particularly polyurethanes, or polymers obtainable by free-radical polymerization. Particularly suitable are polymers obtainable by free-radical polymerization, preferably those composed to an extent of at least 40% by weight, more preferably at least 60% by weight, and very preferably at least 80% by weight, of what are called principal monomers.

The principal monomers are selected from C₁ to C₂₀ alkyl (meth)acrylates, vinyl esters of carboxylic acids comprising up to 20 C atoms, vinylaromatics having up to 20 C atoms, ethylenically unsaturated nitriles, vinyl halides, vinyl ethers of alcohols comprising 1 to 10 C atoms, aliphatic hydrocarbons having 2 to 8 C atoms and 1 or 2 double bonds, or mixtures of these monomers. Examples of preferred monomers are given above. The polymers may be noncrosslinked or crosslinked; crosslinked polymers are obtainable by accompanying use of compounds having at least two copolymerizable ethylenically unsaturated groups, such as those listed above, for example.

### Other Constituents of the Coating Composition

The radiation curable coating composition is preferably liquid at 20° C. and 1 bar. For this purpose, in one particularly preferred embodiment, it comprises no solvents, or virtually no solvents, such as organic solvents or water. The term “solvents” refers here to compounds which are liquid at 20° C. and 1 bar and which, after radiation curing and, where practiced, supplementary thermal curing, must be removed because they are not bound to the resultant polymer film by radiation curing or other chemical reaction.

The radiation curable coating composition preferably comprises less than 10 parts by weight of solvent, more preferably less than 5 parts by weight of solvent, very preferably less than 1 part by weight of solvent, per 100 parts by weight of coating composition. More particularly the radiation curable coating composition comprises substantially no solvent.

The coating composition preferably comprises at least one photoinitiator.

The photoinitiator may be of the kind, for example, known as α-splitters, which are photoinitiators in which a chemical bond is split to produce 2 free radicals which initiate the further crosslinking or polymerization reactions.

Examples include acylphosphine oxides (Lucirin® products from BASF), hydroxyalkylphenones (e.g., Irgacure® 184), benzoin derivatives. benzyl derivatives and dialkyloxyacetophenones.

More particularly they may also be of the type known as H abstractors, which abstract a hydrogen atom from the polymer chain; examples here are photoinitiators having a carbonyl group. This carbonyl group is inserted into a C—H bond to form a C—C—O—H moiety.

Mention may be made here more particularly of acetophenone, benzophenone, and their derivatives.

Mention may also be made of benzoins or benzoin ethers.

Where cationically polymerizable compounds are employed, it is also possible for photoinitiators for the cationic polymerization to be used as well.

Photoinitiators can be used alone or else in a mixture, particular contemplation extending to mixtures of photoinitiators with different modes of action.

Photoinitiators may also be part of a compound which is a constituent of the binder, as for example of an above polymer or of an above crosslinker, and which, in the course of the radiation cure, are bound chemically in this way to the resultant coating.

In the case of a thermal cure or a combination of a radiation cure and a thermal cure, it is possible for one or more thermally activable initiators to be added, such as peroxides, azo compounds, etc.

Besides the binder (compounds of the formula I, other monomers, crosslinkers or other compounds) and, where included, the photoinitiator, the coating composition may comprise further constituents. Those suitable include, in particular, pigments, including effect pigments, dyes, fillers, stabilizers, UV absorbers for example, antioxidants or biocides, flow control assistants, defoamers, wetting agents, antistats, etc.

### The Use

The radiation curable coating composition is used for producing protective or surface coatings. The process of the invention produces substrates coated with a protective or surface coating.

The coating composition may be applied to the desired substrates by customary methods, such as spreading, spraying, dipping, knife coating or printing. The substrates may be substrates with surfaces of wood, paper, card, plastic or metal, for example. The layer thicknesses are generally between a few micrometers and a few millimeters, and may be set by applying the desired amount of coating composition.

In accordance with processes of the invention, the coating is radiation cured. The radiation cure may take place with high-energy electromagnetic radiation, more particularly with UV light or electron beams. In one special embodiment there may additionally be a thermal treatment, in order, for example, to activate thermal initiators and/or to remove solvent.

The coatings obtained have good performance properties; in particular they exhibit high hardness in tandem with high elasticity. The reactivity of the coating compositions is also good, i.e., sufficient curing takes place even in a short time of irradiation with low electromagnetic energy.

The new compounds of the formula I according to the invention are liquid at 20° C. and 1 bar and are particularly suitable for the process of the invention. They are also suitable, however, for other applications, as a constituent of printing inks, for example.

### EXAMPLES

### Preparation Example 1

71 parts of 2-ethyl-2-methyl-1,3-propanediol (EMPD), 95 parts of acrylic acid, 34 parts of cyclohexane, 0.15 part of methylhydroquinone, 0.1 part of copper chloride, and 1.4 parts of sulfuric acid are combined in a multineck flask and heated at reflux at an external temperature of 120° C. During 4 hours of reaction time, 23 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 80 parts of the diacrylate of the alcohol employed, in the form of a clear, yellowish liquid having a viscosity of 20 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

### Preparation Example 2

86 parts of 2-isopropyl-2-methyl-1,3-propanediol (IMPD), 104 parts of acrylic acid, 39 parts of cyclohexane, 0.15 part of methylhydroquinone, 0.1 part of copper chloride, and 1.4 parts of sulfuric acid are combined in a multineck flask and heated at reflux at an external temperature of 120° C. During 4 hours of reaction time, 25 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 80 parts of the diacrylate of the alcohol employed, in the form of a clear, yellowish liquid having a viscosity of 40 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

### Preparation Example 3

96 parts of 2-butyl-2-ethyl-1,3-propanediol (BEPD), 95 parts of acrylic acid, 34 parts of cyclohexane, 0.15 part of methylhydroquinone, 0.1 part of copper chloride, and 1.4 parts of sulfuric acid are combined in a multineck flask and heated at reflux at an external temperature of 120° C. During 4 hours of reaction time, 23 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 105 parts of the diacrylate of the alcohol employed, in the form of a yellowish liquid having a viscosity of 45 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

### Preparation Example 4

113 parts of a mixture of 2-pentyl-2-propyl-1,3-propanediol (PPPD) and 2-(2-methylbutyl)-2-propyl-1,3-propanediol (MBPPD); mixing ratio approximately 10:1 PPPD:MBPPD; 95 parts of acrylic acid, 34 parts of cyclohexane, 0.15 part of methylhydroquinone, 0.1 part of copper chloride, and 1.4 parts of sulfuric acid are combined in a multineck flask and heated at reflux at an external temperature of 120° C. During 4 hours of reaction time, 23 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 120 parts of the diacrylate of the alcohol employed, in the form of a yellow liquid having a viscosity of 70 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

### Preparation Example 5

113 parts of 2-isopropyl-2-(3-methylbutyl)-1,3-propanediol (IMBPD), 95 parts of acrylic acid, 34 parts of cyclohexane, 0.15 part of methylhydroquinone, 0.1 part of copper chloride, and 1.4 parts of sulfuric acid are combined in a multineck flask and heated at reflux at an external temperature of 120° C. During 4 hours of reaction time, 23 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 80 parts of the diacrylate of the alcohol employed, in the form of a brownish yellow liquid having a viscosity of 230 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

### Preparation Example 6

125 parts of 2-methyl-2-phenyl-1,3-propanediol (MPPD), 119 parts of acrylic acid, 50 parts of cyclohexane, 0.2 part of methylhydroquinone, 0.1 part of copper chloride, and 2 parts of sulfuric acid are combined in a multineck flask and heated at reflux at an external temperature of 120° C. During 6 hours of reaction time, 29 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 196 parts of the diacrylate of the alcohol employed, in the form of a clear, yellow-orange liquid having a viscosity of 50 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

### Preparation Example 7

94 parts of 1,1-cyclohexanedimethanol (CHDM), 103 parts of acrylic acid, 40 parts of cyclohexane, 0.2 part of methylhydroquinone, 0.1 part of copper chloride, and 2 parts of sulfuric acid are combined in a multineck flask and heated at reflux at an external temperature of 120° C. During 4 hours of reaction time, 25 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 132 parts of the diacrylate of the alcohol employed, in the form of a clear, yellow-orange liquid having a viscosity of 45 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

### Preparation Example 8

148 parts of 1,1-cyclooctanedimethanol (CODM), 103 parts of acrylic acid, 50 parts of cyclohexane, 0.2 part of methylhydroquinone, 0.1 part of copper chloride, and 2 parts of sulfuric acid are combined in a multineck flask and heated at reflux at an external temperature of 120° C. During 4 hours of reaction time, 28 parts of water are removed by distillation. The reaction product obtained in this way is diluted with further cyclohexane and extracted by shaking with aqueous sodium hydroxide solution. The organic phase is then dried over sodium sulfate. Distillative removal of the solvent leaves 202 parts of the diacrylate of the alcohol employed, in the form of a clear, yellow-orange liquid having a viscosity of 145 mPas (Epprecht cone/plate viscometer (cone B)) at 23° C. The IR spectrum shows virtually no further OH absorption at 3400 cm⁻¹, and an acrylate band at 810 cm⁻¹. The ¹H NMR is in agreement with the expected structure.

Structures of the diacrylates of the above preparation examples P1 to P8,

The diacrylates of preparation examples 1 to 8 are those of the formula II having the following substituents (for all of them it is the case that R³═Y=acryloyl)

P1: R¹=ethyl, R²=methyl, n, m=0 (for short EMPD-DA)
P2: R¹=isopropyl, R²=methyl, n, m=0 (for short IMPD-DA)
P3: R¹=butyl, R²=ethyl, n, m=0 (for short BEPD-DA)
P4: mixture of about 10 parts of R¹=pentyl, R²=methyl, n, m=0 (for short PPPD-DA) and 1 part of R¹=2-methylbutyl, R²=propyl, n, m=0 (for short MBPPD-DA)
P5: R¹=isopropyl, R²=3-methylbutyl, n, m=0 (for short IMBPD-DA)
P6: R¹=methyl, R²=phenyl, n, m=0 (for short MPPD-DA)
P7: R¹ and R² together form a cyclohexane ring; n, m=0

Formula:

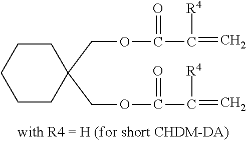

P8: R¹ and R² together form a cyclooctane ring; n, m=0

Formula:

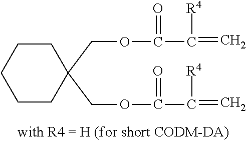

### Coating Compositions

Coating compositions were prepared from 70% by weight of Laromer® 8765 and 30% by weight of the diacrylate specified in the table.

Laromer 8765 is an aliphatic epoxy acrylate with a functionality of 2 that is available commercially from BASF SE. The mixtures of Laromer 8765 and the diacrylate were liquid at room temperature.

The diacrylate used was one of the following diacrylates of the formula II (unalkoxylated, i.e., n, m=0):

for comparison
NPG-DA (neopentylglycol diacrylate, R¹, R²=methyl)
EMPD-DA (R¹=ethyl, R²=methyl)

### Inventive

BEPD-DA (R¹=butyl, R²=ethyl)
mixture of PPPD-DA (R¹=pentyl, R²=methyl) and MBPPD-DA (R¹=2-methylbutyl, R²=propyl)
IMPD-DA (R¹=isopropyl, R²=methyl)
IMBPD-DA (R¹=isopropyl, R²=3-methylbutyl)

### Preparation of the Coating Compositions and Performance Testing

The coating compositions were applied in a layer thickness of approximately 50 μm by means of a slotted doctor blade to glass or Bonder panel substrates and exposed twice in a UV exposure unit equipped with a high-pressure mercury UV lamp having an energy of 120 W/m, the distance from the lamp to the substrate being 10 cm, and the speed of the conveyor belt on which the coated substrates rest and are conveyed beneath the lamp being 10 meters (m)/min.

### Pendulum Damping

The pendulum damping (DIN 53 157) is a measure of the hardness of the coating. It is reported in seconds (s), with high values denoting high hardness.

### Erichsen Cupping

The Erichsen cupping (DIN 53 156) is a measure of the flexibility and elasticity of the coating. For the determination of Erichsen cupping, the coating composition is applied to BONDER panel 132 and exposed as described above. The Erichsen cupping is then determined by impressing a metal ball into the uncoated side of the panel, and determining the depth of impression at which the film ruptures. It is reported in millimeters (mm), with high values denoting high flexibility.

### Chemical Resistance

props of different liquids were applied to the exposed coatings. After an exposure time of 24 hours, inspection takes place to determine whether permanent damage and/or coloration has occurred, which is scored from 0 (=no damage or color) to 5 (strong damage or color).

TABLE 1

| | | Pendulum | Erichsen | Resistance | Resistance |
|---|---|---|---|---|---|
| Example | | damping | cupping | to | to |
| No. | Diacrylate | (s) | (mm) | red wine | coffee |
| C1 | NPG-DA | 91 | 3.4 | 2 | 2 |
| C2 | EMPD-DA | 73 | 3.9 | 2 | 2 |
| I1 | BEPD-DA | 46 | 4.5 | 2 | 2 |
| I2 | PPPD-DA/ | 41 | 4.3 | 2 | 2 |
| | MBPPD-DA | | | | |
| I3 | IMPD-DA | 63 | 4.2 | 2 | 2 |
| I4 | IMBPD-DA | 34 | 4.8 | 2 | 2 |

1. A process for producing a protective or surface coating comprising coating a substrate with a radiation curable coating composition and subsequent radiation curing, wherein the radiation curable coating composition comprises a compound of the formula I 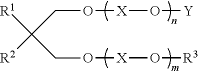
in which
R¹ and R² independently of one another are an organic radical having in each case at least one C atom, but where the sum of the C atoms of R¹ and R² is at least 4, or R¹ and R² together form a ring system comprising at least 4 C atoms,
R³ is a hydrogen atom or Y,
X is a C₁ to C₁₀ alkylene group,
n and m independently of one another are 0 or an integer from 1 to 50,
Y is a group selected from
—CH═CH₂; —CH₂—CH═CH₂;
or 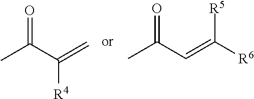
and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

2. The process according to claim 1, wherein the radiation curable coating composition comprises a compound of the formula I where
R¹ and R² independently of one another are an organic radical having in each case at least one C atom, but where the sum of the C atoms of R¹ and R² is at least 4, and R¹ or R², or R¹ and R², comprises or comprise at least one tertiary or quaternary carbon atom,
and R³, R⁴, R⁵, R⁶, Y, n, m, and X have the definition above.

3. The process according to claim 1, wherein the radiation curable coating composition comprises a compound of the formula I where
R¹ and R² independently of one another are an organic radical having in each case at least one C atom, where at least one of the radicals, R¹ or R² is or comprises a ring system,
and R³, R⁴, R⁵, R⁶, Y n, m, and X have the definition above.

4. The process according to claim 1, wherein the radiation curable coating composition comprises a compound of the formula I where
R¹ and R² together form a ring system comprising at least 4 C atoms,
and R³, R⁴, R⁵, R⁶, Y, n, m, and X have the definition above.

5. The process according to claim 1, wherein the radiation curable coating composition comprises a compound of the formula II 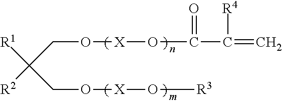
in which
X, R¹, R², R⁴, and n and m have the definition above.

6. The process according to claim 1, wherein the radiation curable coating composition is composed to an extent of 0.1% to 50% by weight of compounds of the formula I.

7. The process according to claim 1, wherein the radiation curable coating composition is composed to an extent of more than 50% by weight of compounds having at least one acryloyl or methacryloyl group ((meth)acryloyl group for short).

8. The process according to claim 1, wherein the radiation curable coating composition is liquid at 20° C. and 1 bar.

9. The process according to claim 1, wherein the radiation curable coating composition comprises less than 10 parts by weight of water per 100 parts by weight of coating composition.

10. The process according to claim 1, wherein the radiation curable coating composition comprises at least one photoinitiator.

11. A substrate coated with a protective or surface coating, obtained by a process according to claim 1.

12. A radiation curable coating composition comprising a compound of the formula I 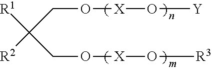
in which
R¹ and R² independently of one another are an organic radical having in each case at least one C atom, but where the sum of the C atoms of R¹ and R² is at least 4, or R¹ and R² together form a ring system comprising at least 4 C atoms,
R³ is a hydrogen atom or Y,
X is a C₁ to C₁₀ alkylene group,
n and m independently of one another are 0 or an integer from 1 to 50,
Y is a group selected from
—CH═CH₂; —CH₂—CH═CH₂;
or 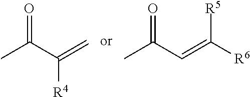
and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group,
but where a compound with R¹=butyl and R²=ethyl is excluded.

13. A compound of the formula I 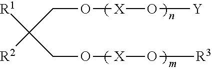
in which
R¹ and R² independently of one another are an organic radical having in each case at least one C atom, but where the sum of the C atoms of R¹ and R² is at least 4 and R¹ or R², or R¹ and R², comprises or comprise at least one tertiary or quaternary carbon atom,
R³ is a hydrogen atom or Y,
X is a C₁ to C₁₀ alkylene group,
n and m independently of one another are 0 or an integer from 1 to 50,
Y is a group selected from
—CH═CH₂; —CH₂—CH═CH₂;
or 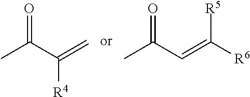
and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

14. A compound of the formula I 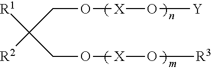
in which
R¹ and R² independently of one another are an organic radical having in each case at least one C atom, where at least one of the radicals, R¹ or R², is or comprises a ring system,
R³ is a hydrogen atom or Y,
X is a C₁ to C₁₀ alkylene group,
n and m independently of one another are 0 or an integer from 1 to 50,
Y is a group selected from
—CH═CH₂; —CH₂—CH═CH₂;
or 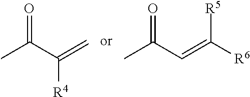
and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

15. A compound of the formula I 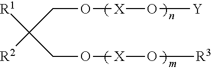
in which
R¹ and R² together form a ring system comprising at least 4 C atoms,
R³ is a hydrogen atom or Y,
X is a C₁ to C₁₀ alkylene group,
n and m independently of one another are 0 or an integer from 1 to 50,
Y is a group selected from
—CH═CH₂; —CH₂—CH═CH₂;
or 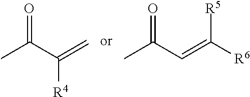
and
R⁴ can be an H atom, a methyl group or —CH₂—C(═O)—OH, and
R⁵ is an H atom and R⁶ is a —C(═O)—OH group or, alternatively,
R⁶ is an H atom and R⁵ is a —C(═O)—OH group.

16. A compound of the formula II 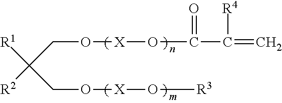
in which
R¹ and R² independently of one another are an organic radical having in each case at least one C atom, but where the sum of the C atoms of R¹ and R² is at least 4 and R¹ or R², or R¹ and R², comprises or comprise at least one tertiary or quaternary carbon atom,
R³ is a hydrogen atom or Y,
X is a C₁ to C₁₀ alkylene group,
n and m independently of one another are 0 or an integer from 1 to 50,
Y is a group 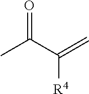
and R⁴ can be an H atom or a methyl group.

17. A compound of the formula II 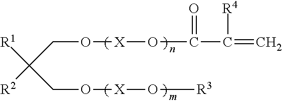
in which
R¹ and R² independently of one another are an organic radical having in each case at least one C atom, where at least one of the radicals, R¹ or R², is or comprises a ring system,
R³ is a hydrogen atom or Y,
X is a C₁ to C₁₀ alkylene group,
n and m independently of one another are 0 or an integer from 1 to 50,
Y is a group 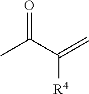
and R⁴ can be an H atom or a methyl group.

18. A compound of the formula II 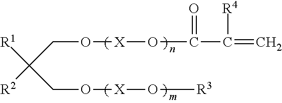
in which
R¹ and R² together form a ring system comprising at least 4 C atoms,
R³ is a hydrogen atom or Y,
X is a C₁ to C₁₀ alkylene group,
n and m independently of one another are 0 or an integer from 1 to 50,
Y is a group 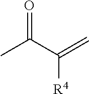
and R⁴ can be an H atom or a methyl group.
